(19) United States
(12) Patent Application Publication
(10) Pub. No.: US 2013/0260340 A1
(43) Pub. Date: Oct. 3, 2013

(54) POWDER FOR ENHANCING FEATURE CONTRAST FOR INTRAORAL DIGITAL IMAGE SCANNING

(71) Applicants: David B. Stegall, Saint Paul, MN (US); Zhisheng Yun, Woodbury, MN (US)

(72) Inventors: David B. Stegall, Saint Paul, MN (US); Zhisheng Yun, Woodbury, MN (US)

(73) Assignee: 3M Innovative Properties Company

(21) Appl. No.: 13/433,643

(22) Filed: Mar. 29, 2012

(51) Int. Cl. A61B 6/14

(52) U.S. Cl. 433/217.1

(57) ABSTRACT

A method for intraoral image scanning using a powder with enhanced feature contrast. The method includes applying the powder to an intraoral structure and using an intraoral scanner in order to obtain electronic digital scan images of the intraoral structure. The powder includes a material providing enhanced feature contrast of the intraoral structure such as black particles combined with a white powder. The scan images can be used to create a 3D digital impression or model of the intraoral structure.

### BACKGROUND

Certain intraoral scanning systems rely upon a powder that is applied to the teeth before video imaging and subsequent three-dimensional (3D) digital impressions or models can be successfully generated. One of the challenges for successfully generating digital impressions using a multiple view geometry method is that a sufficient number of features with sufficient contrast must be obtained in the video images of the teeth. There is a wide range of teeth color and texture in the patient populace in conjunction with practical resolution limitations of the camera system that necessitate the application of a powder to homogenize all possible imaging conditions.

These scanning systems have used a white powder comprised of titanium dioxide particles. The white powder was deemed sufficient to provide the consistent scattering of light from the scanning wand and texture or granularity that would lead to adequate features in the video images. However, an over application of the powder can cause a reduction of contrast available in the image and thus a reduction in the number of features available for the digital impression. Furthermore, there is an uncontrolled level of contrast due to the variability of tooth color in the patient populace.

Although the white titania can be effective at reflecting and scattering of the illuminating light, there is no control over the dark regions of the tooth surface underlying the powder. As a consequence, the powder provides a predictable maximum pixel brightness in any given image but without control over the darkest pixel level. Without controlling the dark portions of an image, there is no predictable contrast level of the images across the patient populace. Thus, there may be many instances where teeth coated with titania powder does not easily provide for adequate surface features for producing a digital oral impression.

### SUMMARY

A method, consistent with the present invention, involves intraoral image scanning using a powder with enhanced feature contrast. The method includes applying a powder to an intraoral structure and scanning the intraoral structure having the applied powder with an intraoral scanner in order to obtain electronic digital images of the structure. The powder includes a material providing for the enhanced feature contrast of the intraoral structure.

### BRIEF DESCRIPTION OF THE DRAWINGS

The accompanying drawings are incorporated in and constitute a part of this specification and, together with the description, explain the advantages and principles of the invention. In the drawings,

FIG. 1 is diagram of an intraoral scanning system;

FIG. 2 is a graph of the sum of the spatial frequency spectrum between 0 and 0.335 cycles/pixel when scanning with only white powder for the comparative Example;

FIG. 3 is a graph of successful correlations when scanning with only white powder for the comparative Example;

FIG. 4 is a graph of the sum of the spatial frequency spectrum between 0 and 0.335 cycles/pixel when scanning with an enhanced contrast powder for Example 1;

FIG. 5 is a graph of successful correlations when scanning with an enhanced contrast powder for Example 1;

FIG. 6 is a graph of the sum of the spatial frequency spectrum between 0 and 0.335 cycles/pixel when scanning with an enhanced contrast powder for Example 2; and

FIG. 7 is a graph of successful correlations when scanning with an enhanced contrast powder for Example 2.

### DETAILED DESCRIPTION

Embodiments of the present invention include a powder for enhancing feature contrast to be applied to teeth for intraoral scanning to generate digital models of the teeth. In particular, light absorbing particles are used with white particles to ensure that regions of the scanned image will have a minimized brightness level. By combining white and light absorbing particles and then coating the mixture onto the teeth, there can be a more predictable contrast level in the video images of the teeth. Regardless of inherent tooth color or thickness of the powder application, the powder provides both bright and dark features on the surface where imaging occurs.

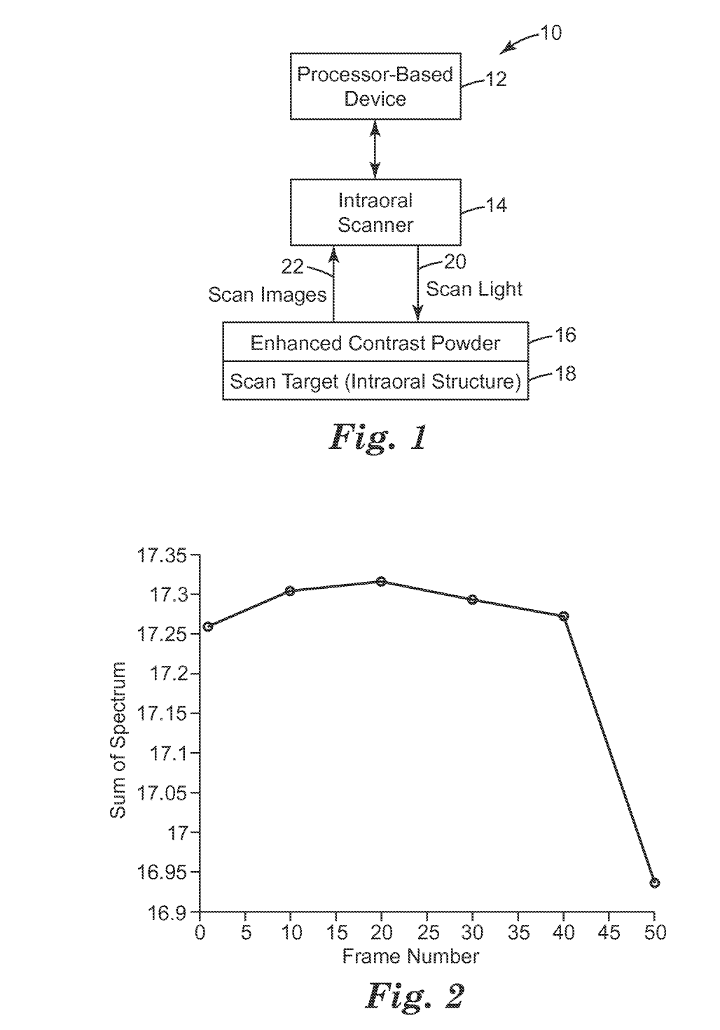

FIG. 1 is diagram of an intraoral scanning system 10. System 10 includes a processor-based device 12 electronically connected with an intraoral scanner 14. In use, intraoral scanner 14 projects a scan light 20 onto a scan target 18 (an intraoral structure) and generates scan images 22 using an optical sensor. An enhanced contrast powder 16 is applied to scan target 18 prior to acquiring the images. Scan images 22 are transmitted to processor-based device 12, which generates a 3D electronic digital impression or model of scan target 18 based upon the images. Scan images 22 can also be combined to create video images of the intraoral structure.

Systems for processing scanned images to generate and display 3D digital models of intraoral structures are described in U.S. Pat. Nos. 7,605,817 and 7,912,257, both of which are incorporated herein by reference as if fully set forth.

Scanning wand constructions for acquiring digital images of intraoral structures for use in generating corresponding 3D digital models are described in U.S. Pat. Nos. 7,746,568 and 7,646,550, both of which are incorporated herein by reference as if fully set forth.

One particular enhanced contrast powder, as described in the Examples, includes a combination of white powder with dark-color particles, such as black particles. When this powder is applied to the teeth, the intraoral scanner can obtain more features in any given image with which to generate the digital model. In particular, the scan images exhibit an increased number of high contrast features on the surface of the teeth that are essential to generating disparity maps that precede meshing and digital impression maps to create the 3D model. As a consequence, successful scans can be generated more often. Since any given video frame has more features available for producing the mesh, the scan can proceed more quickly and with more redundancy of mesh points, which can prove useful for the accuracy of the scan.

Another enhanced contrast powder includes a premix of white powder with a dark-color powder such as black particles, as described in the Examples. The dark-color powder for the premix can also include other dark-color powders, such as a dark blue, dark green, or others. The volume ratio for the white and dark-color premix can be 1:5 (white:dark-color), a ratio between 1:4 and 1:1 inclusive (white:dark-color), or 5:1 (white:dark-color), 4:1, or a ratio between 3:1 and 1:1 inclusive (white:dark-color). The preferred contrast is 1:1 (white:dark-color) in volume.

Enhanced contrast powders can also include other materials added to a white powder.

The other materials can provide enhanced feature contrast by substantially absorbing light within the spectral range of the scan light from the intraoral scanner. The other materials can also provide enhanced feature contrast by having a color different from the powder.

Other types of medical grade intraoral high contrast white powders, aside from TiO₂ as used in the Examples, can alternatively be used. Also, the dark-color powder does not need to be black. It only needs to not strongly reflect or scatter the incident light from the scanner. For example, a yellow or red particle, as seen under white light, would appear very dark under a scanner using blue light.

### EXAMPLES

These Examples are merely for illustrative purposes only and are not meant to be limiting on the scope of the appended claims.

### Comparative Example

A powder gun was used to apply the white powder (TiO₂) to a typodont. Using an intraoral camera operating in the same manner as the LAVA Chairside Oral Scanner (3M Company, St. Paul, Minn.), a 50 frame video was then taken while moving the typodont away from the camera. The first frame started about 5 mm from the camera and the fiftieth frame was a few millimeters further away. To capture the video, the camera was held in an immobile fixture looking down on the typodont. The typodont rested on a lab jack with a level surface that could be translated vertically to control the distance between the typodont and camera. The video began with the closest surface of the typodont being 5 mm from the camera and by the 50th frame, the lab jack had been translated downward to increase the distance by several more millimeters.

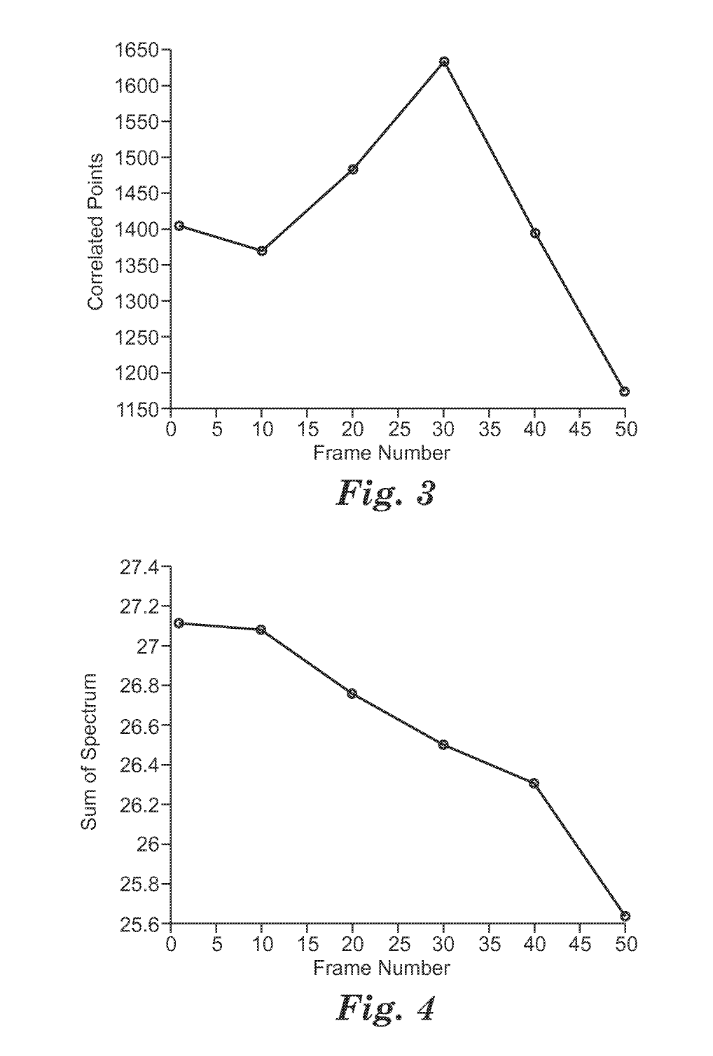

FIG. 2 is a graph of the sum of the spatial frequency spectrum when scanning with only the white powder. FIG. 3 is a graph of successful correlations when scanning with only the white powder.

### Example 1

Example 1 used black powder as the material for the enhanced feature contrast. A powder gun was used to apply the white powder (TiO₂) to a typodont, and the black powder (activated charcoal) was then lightly sprinkled over a heavily white powdered typodont. Using the same camera as used for the Comparative Example, a 50 frame video was then taken while moving the typodont away from the camera in the same manner as provided for the Comparative Example. The first frame started about 5 mm from the camera and the fiftieth frame was a few millimeters further away.

FIG. 4 is a graph of the sum of the spatial frequency spectrum when scanning with this enhanced contrast powder. Comparing FIG. 4 with FIG. 2 indicates that significantly more information was obtained when black powder was applied onto the white powder than when using only the white powder. The improvement was due to a marked improvement in the inherent object feature contrast.

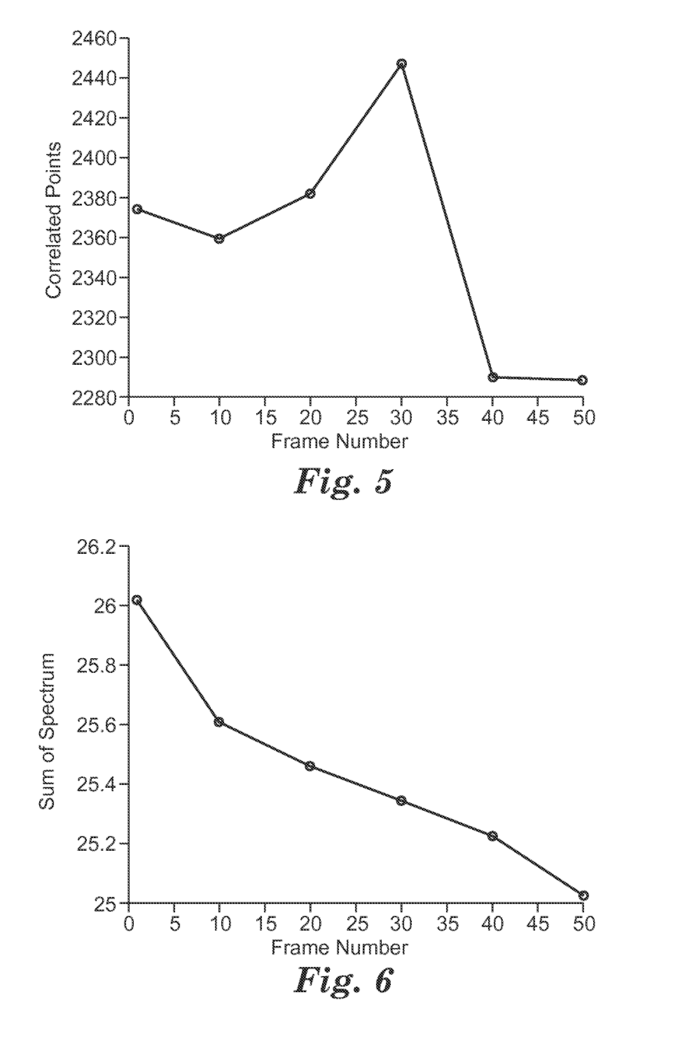

FIG. 5 is a graph of successful correlations when scanning with this enhanced contrast powder. As shown by comparing FIG. 5 with FIG. 3, the improvement in finer image content using this enhanced powder translated into a much greater number of correlations within the image than when using only the white powder. The correlation algorithm divided the original 768×1024 pixel image into 64×96 regions for correlation analysis. Thus, there were a maximum of 6144 correlations. Relative improvements resulting from using this enhanced contrast powder ranged between 49% and 94% depending upon the frame.

### Example 2

Example 2 also used black powder as the material for the enhanced feature contrast, except that the carbon black was mixed with the white powder (TiO₂) to produce a mixture (premix) with a gray hue. A powder gun was used to apply this mixture to a typodont. Using the same camera as used for the Comparative Example, a 50 frame video was then taken while moving the typodont away from the camera in the same manner as provided for the Comparative Example. The first frame started about 5 mm from the camera and the fiftieth frame was a few millimeters further away.

FIG. 6 is a graph of the sum of the spatial frequency spectrum when scanning with this enhanced contrast powder. Comparing FIG. 6 with FIG. 2 indicates that significantly more information was obtained with use of the premix than when using only the white powder.

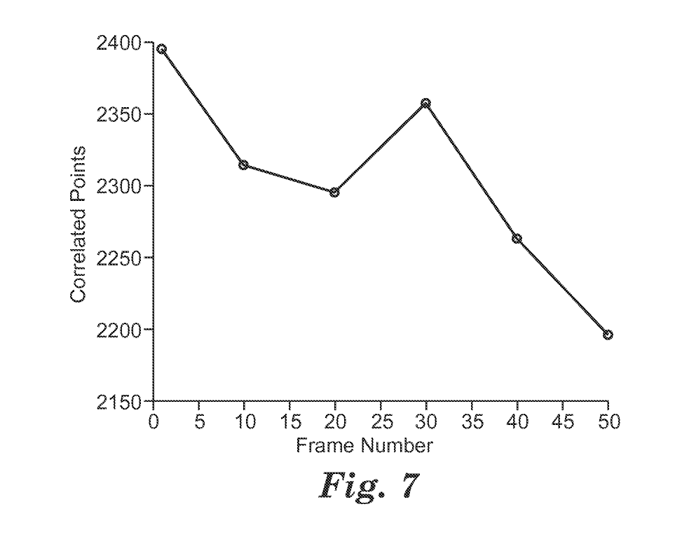

FIG. 7 is a graph of successful correlations when scanning with this enhanced contrast powder. As shown by comparing FIG. 7 with FIG. 3, the use of the premix resulted in a greater number of correlations within the image than when using only the white powder.

1. A method for intraoral image scanning using a powder with enhanced feature contrast, comprising the steps of:
applying a powder to an intraoral structure, wherein the powder includes a material providing enhanced feature contrast of the intraoral structure, wherein the applying step comprises first applying a white powder and then applying a dark-color powder onto the white powder while leaving portions of the white powder exposed;
projecting a scan light to the intraoral structure, wherein the scan light has a spectral range less than wavelengths of visible light; and
scanning the intraoral structure having the applied powder and illuminated by the projected scan light with an intraoral scanner in order to obtain electronic digital images of the intraoral structure,
wherein the dark-color powder absorbs light within the spectral range of the scan light in order to enhance feature contrast of the intraoral structure.

2-3. (canceled)

4. The method of claim 1, wherein the white powder comprises a medical grade intraoral high contrast white powder.

5. (canceled)

6. The method of claim 1, wherein the dark-color powder comprises a black powder.

7-9. (canceled)

10. The method of claim 1, wherein the applying step comprises using a powder gun to apply the powder.

11. The method of claim 1, wherein the spectral range of the scan light is blue light.

12. The method of claim 11, wherein the dark-color powder comprises a yellow or red powder.

13. The method of claim 11, wherein the dark-color powder comprises a non-black powder that provides the enhanced feature contrast.
